(19) United States
(12) Patent Application Publication
(10) Pub. No.: US 2016/0038235 A1
(43) Pub. Date: Feb. 11, 2016

(54) INGENOL MEBUTATE IN COMBINATION WITH LASER THERAPY FOR THE TREATMENT OF HYPERKERATOTIC SKIN LESIONS

(71) Applicant: LEO LABORATORIES LIMITED, Dublin (IE)

(72) Inventors: Merete HÆDERSDAL, Copenhagen NV (DK); Andres ERLANDSSON, Copenhagen NV (DK); André Huss ERIKSSON, Ballerup (DK); John Robert ZIBERT, Ballerup (DK)

(73) Assignee: LEO LABORATORIES LIMITED, Dublin (IE)

(21) Appl. No.: 14/781,824

(22) PCT Filed: Apr. 2, 2014
(86) PCT No.: PCT/EP2014/056609

§ 371 Date: Oct. 1, 2015

(30) Foreign Application Priority Data
Apr. 2, 2013 (DK) PA 2013 00195

(51) Int. Cl. A61B 18/20; A61K 9/00; A61K 9/06; A61K 31/22

(52) U.S. Cl. CPC A61B 18/20; A61K 31/22; A61K 9/0014; A61K 9/06

(57) ABSTRACT

The invention relates to the treatment of actinic keratosis (AK) lesions using laser therapy followed by treatment with ingenol mebutate (e.g., PEP005 Gel).

### FIELD OF THE INVENTION

The invention relates to the treatment of hyperkeratotic skin lesions using sequential laser therapy and field treatment with ingenol mebutate (e.g., PEP005 Gel).

### BACKGROUND OF THE INVENTION

Hyperkeratosis may result in decreased penetration of topically applied drugs. Hyper keratosis may be found in the pathological conditions such as warts (genital and non-genital), actinic keratosis (AK), squamous cell carcinoma, basal cell carcinoma, and malignant melanoma. AK is a common skin condition visible as thickened, cornified, scaly lesions and characterised histologically by atypical epithelial proliferation. Actinic keratoses usually develop on areas that are frequently exposed to the sun (e.g., face, ears, lips, scalp, neck, forearms, and back of the hands). It is estimated that AK occurs in 11-50% of the population aged 40 and older in the US and Australia. In Europe the prevalence rate is from 11-25% for people aged 40 or older. Patients with AK tend to have Fitzpatrick type I or II skin (fair skin) which burns and does not tan.

In the context of AK, field cancerisation is characterized by the epithelial surface of the photodamaged area being susceptible to the development of additional AKs or a malignancy. This is evident by the presence of multiple subclinical and clinically visible AK lesions as well as multifocal preneoplastic changes with genetic mutations. There is also increasing evidence that AK represents squamous cell carcinoma (SCC) in situ in its earliest stages. If left untreated, AK may progress to SCC, with significant morbidity and potential death.

Ingenol mebutate is an ingenol derivative extracted from Euphorbia peplus (E. peplus), a member of the Spurge family. Ingenol mebutate was identified as the principal active component responsible for the selective cytotoxic effects of E. peplus sap, based on its antitumor effects both in vitro and in vivo. Ingenol mebutate is distinguished from current therapeutic options by a substantially shorter duration of treatment (2 to 3 days) compared to approved topical AK products.

It has been noted in the literature that destructive procedures such as dermabrasion, chemical peeling, and laser resurfacing therapeutically ablate the epidermis, therewith also remove aberrant cells such as actinic keratosis. Although laser therapy may be a therapy for AK, treatment settings are not standardised and this is reflected in a wide range of efficacy results. Although short term efficacy with laser therapy demonstrates some good clearance of individual lesions, recurrence rates are high. In addition, the lesion directed laser therapy; fail to address the issue of actinic field cancerisation in patients with AK.

It is an object of the present invention to provide a treatment of actinic keratosis lesions, which provides as well a lesion as a field directed treatment.

### SUMMARY OF THE INVENTION

The invention provides a method for treating hyperkeratotic skin lesions in a subject in need thereof comprising a combination of laser therapy and topical treatment with ingenol mebutate.

The invention also provides Ingenol mebutate in combination with laser therapy for the treatment of hyperkeratotic skin lesions.

The invention also provides a combination treatment comprising laser therapy and ingenol mebutate for treating hyperkeratotic skin lesions.

The invention also provides the use of ingenol mebutate in combination with laser therapy for the treatment of hyperkeratotic skin lesions.

### BRIEF DESCRIPTION OF THE DRAWINGS

FIG. 1 shows the uptake of ingenol mebutate in the different parts of the skin by 50 μm micropores and at different densities.

FIG. 2 shows the uptake of ingenol mebutate in the different parts of the skin by 500 μm micropores and at different densities.

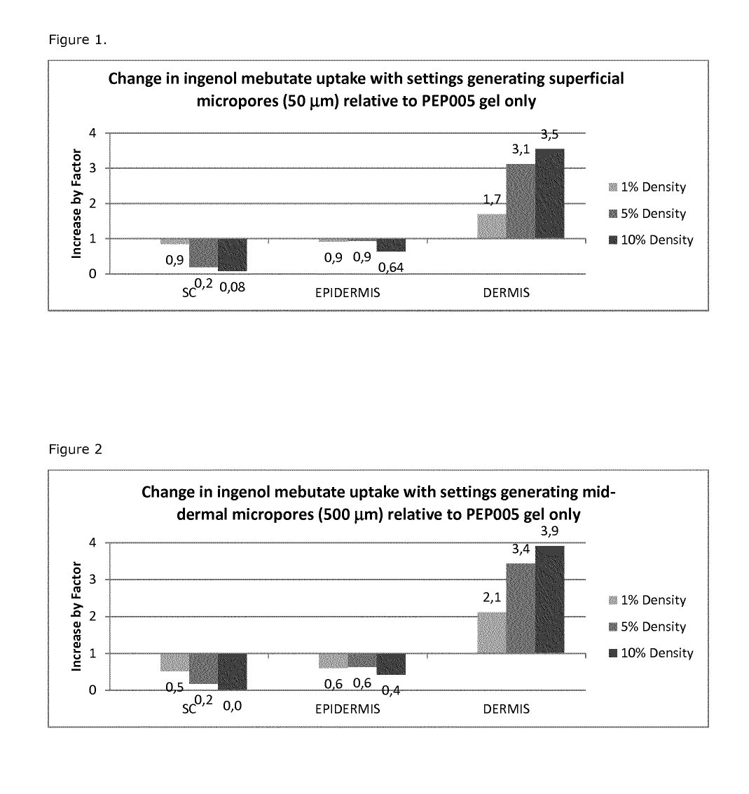

### DETAILED DESCRIPTION OF THE INVENTION

The invention provides a method for treating hyperkeratotic skin lesions in a subject in need thereof, comprising applying laser therapy to the lesion, followed by topical application of ingenol mebutate.

The invention provides a method according to the embodiments above, wherein the ingenol mebutate is applied as a field therapy covering maximum of 25 cm².

The invention provides a method according to any of the embodiments above, wherein the subject is treated by ingenol mebutate on the laser treated lesion at least once.

The invention provides a method according to any of the embodiments above, wherein the subject is treated with 0.015% ingenol mebutate on face or scalp for 3 consecutive days.

The invention provides a method according to any of the embodiments above, wherein the subject is treated with 0.05% ingenol mebutate on trunk and extremities, for 2 consecutive days.

In an embodiment of the invention, reduction in the number of lesions in the treated area of the skin of the subject, is observed after the combination treatment.

In an embodiment of the invention, complete clearance of lesions in the treated area of the skin of the subject, defined as the proportion of subjects with no clinically visible lesions in the selected treatment area, is observed after the combination treatment.

In an embodiment of the invention, the number of clinically visible lesions in the treated area of the skin of the subject is reduced.

It is an object of the present invention to improve the rate of complete clearance of lesions using sequential laser therapy and field treatment with ingenol mebutate.

In embodiments of the invention the hyperkeratotic lesion is actinic keratosis. In an embodiment the hyperkeratotic lesion is a wart. In an embodiment of the invention the hyperkeratotic lesion is a genital wart. In an embodiment of the invention the hyperkeratotic lesion is a non-genital wart. In an embodiment of the invention the hyperkeratotic lesion is squamous cell carcinoma. In an embodiment of the invention the hyperkeratotic lesion is basal cell carcinoma. In an embodiment of the invention the hyperkeratotic lesion is malignant melanoma.

The invention is useful for treating hyperkeratotic skin lesions, and in particular squamous cell carcinoma, basal cell carcinoma, malignant melanoma, warts (genital and non-genital) and actinic keratosis (also called “solar keratosis” or “senile keratosis”). Actinic keratoses may be divided into the following types: hyperkeratotic; pigmented; lichenoid; and atrophic. The invention is particularly suitable for treating hyperkeratotic actinic keratosis, which can be difficult to treat with topical treatment alone. Hyperkeratosis refers to hypertrophy of the horny layer of the skin. The lesions are difficult to treat and the preferred treatment presently is curettage or cryo surgery, which presents some risks of complications as well as scarring. The location of the lesions could also present a treatment issue.

In the context of the present invention an actinic keratosis lesion includes all of the above types of actinic keratosis. In the context of the present invention a particular embodiment is hyperkeratotic actinic keratosis.

In embodiments of the invention, for the hyperkeratotic lesions are warts or genital warts. In embodiments of the invention the hyperkeratotic lesion is a non-melanoma skin cancer. In embodiments of the invention these are plate cell carcinomas. In embodiments of the invention, the cancers are basal cell carcinoma, such as nodular basal cell carcinoma. In embodiment of the invention the lesions are squamous cell carcinoma. In embodiments of the invention, the cancers are malignant melanoma.

In the context of the present invention, laser therapy comprises applying a laser beam to the skin surface. In embodiments the laser is Ablative Fractioned laser type. In an embodiment according to any of the embodiments above, the density of the laser drilled micropores is up to 20%. In an embodiment the density of the laser drilled micropores is up to 15%. In an embodiment the density of the laser drilled micropores is up to 10%. In an embodiment the density of the laser drilled micropores is up to 1%.

In embodiments of the invention the laser therapy comprises drilling micropores in the lesion of up to 500 μm depth. In embodiments of the invention the laser therapy comprises drilling micropores in the lesion of up to 100 μm depth. In embodiments of the invention the laser therapy comprises drilling micropores in the lesion of up to 50 gm depth.

In the context of the present invention the expression “up to” and “maximum” may include the upper and lower limit in the interval.

In embodiments of the invention the delivery of ingenol mebutate to dermis shows up to approximately 4 fold increase following laser pre-treatment compared to no laser pre-treatment. In an embodiment a density dependent 1.7-3.9 fold increase following laser pre-treatment compared to no laser pre-treatment was shown.

The present invention provides as well a lesion directed as a field directed therapy. By applying the laser therapy followed by ingenol mebutate, the specific lesions are treated.

By applying ingenol mebutate topically over the lesion and the field, a treatment of the subclinical lesions is achieved. This is particular important for treating actinic field damage.

For treatment of AK, ingenol mebutate is presently approved by regulatory authorities for treating of face and scalp in a 0.015% concentration over 3 consecutive days. For body and trunk application over 2 consecutive days in a concentration of 0.05% is approved. The pharmaceutical is formulated into a gel which is described in WO 07/68963. Similar dosage regimens are applied in the present invention.

The results obtained by the present invention shows an improved uptake in the epidermis of up to approximately 4 times the uptake obtained without use of the laser. The dosage applied to the lesions by ingenol mebutate can be lowered proportionately compared to the presently used dose for the relevant body parts. Alternatively, the application can be administered only once with either the 0.015% or 0.05% dosage. The combination treatment will probably diminish the side effects of the treatment while obtaining same clearance. Side effects described for treatment of actinic keratosis with ingenol mebutate include local skin reactions. In embodiments of the invention, the field around the lesion can be treated by the standard treatment as approved presently or laser therapy can be applied in the whole field being treated. The size of the field is maximum 25 cm².

The present invention discloses a therapy with laser followed by a topical treatment with ingenol mebutate. The application of a laser beam to fractions of the skin surface results in a microscopic treatment zone (MTZ) consisting of central microscopic ablation zones (MAZ) that are surrounded by a thin layer of carbonixation (eschar) encased by a coagulation zone (microscopica coagulation zone (MCZ)), the area of the residual thermal damage. Owing to different wavelengths and water absorption intensities, the relative ablative and coagulative tissue response varies in terms of the extent of ablation and coagulation between the laser types. However, on the overall microscopic level, the lesions created with these fractionated lasers are comparable, consisting of MAZs and MCZs. There is a diversity of available devices regarding scanning techniques and technical parameters in terms of intensity(W), pulse duration(ms), spot size, spot energy(mJ/pulse) and number of MTZs per cm². The dimension of a given MTZ is mainly determined by laser type, beam configuration and technical parameters. The diameter of the microbeam defines the spot size, which directly reflects the diameter of the MAZ, whereas the penetration depth mainly depends on the energy applied (mJ/pulse).

The laser device itself is Ablative Fractioned laser type which can be of different types such as CO₂ laser, Erbium laser and YSGG lasers.

In embodiments of the present invention the application of the laser therapy to the skin makes micropores in the skin of different depths. Presently, the tested depth of the micropores has been 50 μm and 500 μm. Dependent on the lesions to be treated the depth of the micropores can be individually adjusted.

The density of the micropores over the applied area can be varied. In the present experiments the densities have been selected as 1%, 5% and 10%. In embodiments of the invention the 5% density is preferred as 10% density didn't significantly increase the drug delivery to the skin compartments.

In embodiments of the invention the 50 μm micropore depth is preferred, as the deeper penetration didn't significantly increase delivery of drug to the skin compartments.

The invention thus provides the following embodiments:
1. A method for treating a hyperkeratotic lesion in a subject in need thereof, the method comprising administering a combination of laser therapy and topical treatment with ingenol mebutate to the subject.
2. The method according to embodiment 1, wherein the hyperkeratotic lesion is squamous cell carcinoma, basal cell carcinoma, malignant melanoma, warts (genital and non-genital) and actinic keratosis.
3. The method according to embodiment 1, comprising applying laser therapy to the hyperkeratotic lesion of the subject, followed by topical application of ingenol mebutate to the skin of the subject.
4. The method according to any of the embodiments above, wherein the ingenol mebutate is applied as a field therapy covering maximum of 25 cm².
5. The method according to any of the embodiments above wherein the laser therapy is drilling micropores at a depth of up to 500 μm, such as up to 100 μm, such as up to 50 μm
6. The method according to any of the embodiments above wherein the laser therapy is drilling micropores at a density of up to 10%, such as up to 5%, such as up to 1%.
7. The method of any of the embodiments above, wherein the topical treatment with ingenol mebutate comprises a pharmaceutical formulation of ingenol mebutate.
8. The method of embodiments above, wherein the pharmaceutical formulation of ingenol mebutate is a gel.
9. The method of any of the embodiments above, wherein ingenol mebutate is applied in a concentration of 0.015% or 0.05%.
10. Ingenol mebutate in combination with laser therapy for the treatment of hyperkeratotic skin lesions.
11. Ingenol mebutate in combination with laser therapy according to embodiment 10, wherein ingenol mebutate is applied topically.
12. Ingenol mebutate in combination with laser therapy according to any of embodiments 10-11, wherein the hyperkeratotic skin lesions are squamous cell carcinoma, basal cell carcinoma, malignant melanoma, warts (genital and non-genital) and actinic keratosis.
13. Ingenol mebutate in combination with laser therapy according to any of embodiments 10-12, wherein the laser therapy is followed by topical application of ingenol mebutate to the skin of the subject.
14. Ingenol mebutate in combination with laser therapy according to any of embodiments 10-13, wherein the ingenol mebutate is applied as a field therapy covering maximum of 25 cm².
15. Ingenol mebutate in combination with laser therapy according to any of embodiments 10-14, wherein the laser therapy is drilling micropores at a depth of up to 500 μm, such as up to 100 μm, such as up to 50 μm in the hyperkeratotic skin lesion.
16. Ingenol mebutate in combination with laser therapy according to any of embodiments 10-15, wherein the laser therapy is drilling micropores at a density of up to 10%, such as up to 5%, such as up to 1% in the hyperkeratotic skin lesions.
17. Ingenol mebutate in combination with laser therapy according to any of embodiments 10-16, wherein the topical treatment with ingenol mebutate comprises topical treatment with a pharmaceutical formulation of ingenol mebutate.
18. Ingenol mebutate in combination with laser therapy according to any of embodiments 10-17, wherein the pharmaceutical formulation of ingenol mebutate is a gel.
19. Ingenol mebutate in combination with laser therapy according to any of embodiments 10-18, wherein ingenol mebutate is applied in a concentration of 0.015% or 0.05%.
20. A combination comprising laser therapy and topically applied ingenol mebutate for treating hyperkeratotic skin lesions.
21. The combination according to embodiment 20, wherein the hyperkeratotic skin lesion is squamous cell carcinoma, basal cell carcinoma, malignant melanoma, warts (genital and non-genital) and actinic keratosis.
22. The combination according to any of the embodiments 20-21, wherein the laser therapy is followed by topical application of ingenol mebutate to the skin of the subject.
23. The combination according to any of embodiments 20-22, wherein the ingenol mebutate is applied as a field therapy covering maximum of 25 cm².
24. The combination according to any of embodiments 20-23, wherein the laser therapy is drilling micropores at a depth of up to 500 μm, such as up to 100 μm, such as up to 50 μm.
25. The combination according to any of embodiments 20-24, wherein the laser therapy is drilling micropores at a density of up to 10%, such as up to 5%, such as up to 1%.
26. The combination according to any of embodiments 20-25, wherein the topical treatment with ingenol mebutate comprises a pharmaceutical formulation of ingenol mebutate.
27. The combination according to any of embodiments 20-26, wherein the pharmaceutical formulation of ingenol mebutate is a gel.
28. The combination according to any of embodiments 20-27, wherein ingenol mebutate is applied in a concentration of 0.015% or 0.05%.
29. Use of ingenol mebutate in combination with laser therapy for the treatment of hyperkeratotic skin lesions.
30. The use according to embodiment 29, wherein the hyperkeratotic skin lesion is squamous cell carcinoma, basal cell carcinoma, malignant melanoma, warts (genital and non-genital) and actinic keratosis.
31. The use according to any of the embodiments 29-30, comprising applying laser therapy to the lesion of the subject, followed by topical application of ingenol mebutate to the skin of the subject.
32. The use according to any of the embodiments 29-31, wherein the ingenol mebutate is applied as a field therapy covering maximum of 25 cm².
33. The use according to any of the embodiments 29-32, wherein the laser therapy is drilling micropores at a depth of up to 500 μm, such as up to 100 μm, such as up to 50 μm.
34. The use according to any of the embodiments 29-33, wherein the laser therapy is drilling micropores at a density of up to 10%, such as up to 5%, such as up to 1%.
35. The use according to any of the embodiments 29-34, wherein the topical treatment with ingenol mebutate comprises a pharmaceutical formulation of ingenol mebutate.
36. The use according to any of the embodiments 29-35, wherein the pharmaceutical formulation of ingenol mebutate is a gel.
37. The use according to any of the embodiments 29-36, wherein ingenol mebutate is applied in a concentration of 0.015% or 0.05%.

### EXAMPLES

Penetration of Ingenol mebutate was investigated in a static in vitro Franz cell model, using porcine skin. Prior to ingenol mebutate application, the skin was pre-treated with a fractional 2940 Er:YAG laser. Two settings were used based on pulse durations at 125 or 225 μs, and power at 1.3 or 1.7 W, delivering total energies per laser channel at 11 or 128 mJ, respectively. The settings were investigated at 1, 5 and 10% densities. The shape and depth of the micropores were evaluated in a dissecting microscope. After 21 h in the Franz cells, stratum corneum was tape stripped and liquid chromatography-mass spectrometry (LC-MS) was used to analyze IngMeb concentrations in stratum corneum, skin and receptor fluid.

### Results

TABLE 1

| | | | | | Enhancement | |
|---|---|---|---|---|---|---|
| Intervention | N | Compartment | Uptake (IQR) | | Factor | P-value* |
| 1—No Laser | 9 | | | | | |
| | | Viable skin | 1114 | (1020-1342) | 1 | — |
| | | SC | 708 | (622-865) | | |
| | | Epidermis | 606 | (538-790) | | |
| | | Dermis | 508 | (348-760) | | |
| | | Receiver | 0 | (0-131) | | |
| 2—Superficial 1% | 10 | | | | | |
| | | Viable skin | 1340 | (1205-1625) | 1.20 | 0.142 |
| | | SC | 603 | (484-829) | | |
| | | Epidermis | 558 | (469-653) | | |
| | | Dermis | 843 | (554-982) | | |
| | | Receiver | 122 | (87-190) | | |
| 3—Superficial 5% | 9 | | | | | |
| | | Viable skin | 2152 | (2079-2381) | 1.93 | <0.0001 |
| | | SC | 135 | (107-154) | | |
| | | Epidermis | 568 | (448-805) | | |
| | | Dermis | 1584 | (1511-1739) | | |
| | | Receiver | 408 | (317-634) | | |
| 4—Superficial 10% | 10 | | | | | |
| | | Viable skin | 2189 | (2123-2331) | 1.97 | <0.0001 |
| | | SC | 59 | (26-180) | | |
| | | Epidermis | 388 | (279-550) | | |
| | | Dermis | 1801 | (1750-1900) | | |
| | | Receiver | 547 | (427-679) | | |
| 5—Mid-Dermal-1% | 9 | | | | | |
| | | Viable skin | 1510 | (1358-1760) | 1.35 | 0.024 |
| | | SC | 369 | (248-645) | | |
| | | Epidermis | 366 | (296-560) | | |
| | | Dermis | 1076 | (915-1322) | | |
| | | Receiver | 304 | (215-599) | | |
| 6—Mid-Dermal 5% | 9 | | | | | |
| | | Viable skin | 2126 | (1926-2226) | 1.91 | <0.0001 |
| | | SC | 127 | (35-245) | | |
| | | Epidermis | 386 | (315-418) | | |
| | | Dermis | 1744 | (1515-1899) | | |
| | | Receiver | 677 | (522-916) | | |
| 7—Mid-Dermal 10% | 11 | | | | | |
| | | Viable skin | 2224 | (1628-2436) | 2.00 | <0.0001 |
| | | SC | 9 | (5-18) | | |
| | | Epidermis | 260 | (196-302) | | |
| | | Dermis | 1986 | (1338-2080) | | |
| | | Receiver | 622 | (253-750) | | |

IQR = Interquartile range,
*Change compared to untreated skin (No laser).

With the two settings, superficial and mid-dermal, cone shaped pores were created with depths of approximately 50 and 500 μm respectively. Results from the LC-MC analysis (n=9 for each condition) show that:
With laser pre-treatment compared with no laser pre-treatment less ingenol mebutate was accumulated in stratum corneum (SC) and the accumulation gradually decreased with increasing density. This was seen with both 50 μm and 500 μm micropores. However, at 1% density at 50 μm micropores similar ingenol mebutate concentrations were observed.
In the epidermis it was evident that laser pre-treatment 1 and 5% densities at 50 μm micropores compared with no laser pre-treatment similar amounts of ingenol mebutate was accumulated. 10% density at 50 μm micropores resulted in decreased ingenol mebutate in the epidermis, which was also the case for all densities at 500 μm micropores.
In the dermis a density dependent (1-10%) 1.7-3.9 fold increase in ingenol mebutate was observed with laser pre-treatment compared with no laser pre-treatment. However, no added benefit was observed at 10% density compared with 5%, and with 500 μm compared with 50 μm micropores.

### Conclusion

The above data suggest that pre-treatment with laser enhances the uptake of ingenol mebutate in the skin. Superficial micropores at 5% density are likely to add the safest and enhanced delivery of PEP005 gel. This might enable treatment of hyperkeratotic lesions as well as increase overall efficacy when treating for example AK's with ingenol mebutate.

1. A method for treating a hyperkeratotic lesion in a subject in need thereof, the method comprising administering a combination of laser therapy and topical treatment with ingenol mebutate to the subject.

2. The method according to claim 1, wherein the hyperkeratotic lesion is squamous cell carcinoma, basal cell carcinoma, malignant melanoma, warts (genital and non-genital) and actinic keratosis.

3. The method according to claim 1, comprising applying laser therapy to the lesion of the subject, followed by topical application of ingenol mebutate to the skin of the subject.

4. The method according to claim 1, wherein the ingenol mebutate is applied as a field therapy covering maximum of 25 cm².

5. The method according to claim 1, wherein the laser therapy is drilling micropores at a depth of up to 500 □m, such as up to 100 □m, such as up to 50 □m followed by ingenol mebutate treatment.

6. The method according to claim 1, wherein the laser therapy is drilling micropores at a density of up to 10%, such as up to 5%, such as up to 1%, followed by ingenol mebutate treatment.

7. The method according to claim 1, wherein the topical treatment with ingenol mebutate comprises a pharmaceutical formulation of ingenol mebutate.

8. The method of claim 7, wherein the pharmaceutical formulation of ingenol mebutate is a gel.

9. The method according to claim 2, comprising applying laser therapy to the lesion of the subject, followed by topical application of ingenol mebutate to the skin of the subject.

10. The method according to claim 2, wherein the ingenol mebutate is applied as a field therapy covering maximum of 25 cm².

11. The method according to claim 3, wherein the ingenol mebutate is applied as a field therapy covering maximum of 25 cm².

12. The method according to claim 2, wherein the laser therapy is drilling micropores at a depth of up to 500 □m, such as up to 100 □m, such as up to 50 □m followed by ingenol mebutate treatment.

13. The method according to claim 3, wherein the laser therapy is drilling micropores at a depth of up to 500 □m, such as up to 100 □m, such as up to 50 □m followed by ingenol mebutate treatment.

14. The method according to claim 4, wherein the laser therapy is drilling micropores at a depth of up to 500 □m, such as up to 100 □m, such as up to 50 □m followed by ingenol mebutate treatment.

15. The method according to claim 2, wherein the laser therapy is drilling micropores at a density of up to 10%, such as up to 5%, such as up to 1%, followed by ingenol mebutate treatment.

16. The method according to claim 3, wherein the laser therapy is drilling micropores at a density of up to 10%, such as up to 5%, such as up to 1%, followed by ingenol mebutate treatment.

17. The method according to claim 4, wherein the laser therapy is drilling micropores at a density of up to 10%, such as up to 5%, such as up to 1%, followed by ingenol mebutate treatment.

18. The method according to claim 5, wherein the laser therapy is drilling micropores at a density of up to 10%, such as up to 5%, such as up to 1%, followed by ingenol mebutate treatment.

19. The method according to claim 2, wherein the topical treatment with ingenol mebutate comprises a pharmaceutical formulation of ingenol mebutate.

20. The method according to claim 3, wherein the topical treatment with ingenol mebutate comprises a pharmaceutical formulation of ingenol mebutate.
